(19) United States
(12) Patent Application Publication
(10) Pub. No.: US 2016/0129233 A1
(43) Pub. Date: May 12, 2016

(54) Three dimensional imaging ultrasound with microbubbles to enhance reflow in ST elevation myocardial infarction

(71) Applicant: Andrew Kenneth Hoffmann, Burnaby (CA)

(72) Inventor: Andrew Kenneth Hoffmann, Burnaby (CA)

(21) Appl. No.: 14/757,093

(22) Filed: Nov. 17, 2015

Related U.S. Application Data
(63) Continuation of application No. 13/986,252, filed on Apr. 17, 2013.
Application No. 13/986,252 is a continuation-in-part of application No. 12/798,437, filed on Apr. 5, 2010, now Pat. No. 8,870,796.
Application No. 12/798,437 is a continuation-in-part of application No. 12/291,128, filed on Nov. 5, 2008, now abandoned.
Application No. 12/291,128 is a continuation-in-part of application No. 12/218,054, filed on Jul. 11, 2008, now Pat. No. 8,734,368.
Application No. 12/218,054 is a continuation-in-part of application No. 11/036,386, filed on Jan. 18, 2005, now abandoned.
Application No. 11/036,386 is a continuation-in-part of application No. 10/902,122, filed on Jul. 30, 2004, now Pat. No. 7,517,328.

(30) Foreign Application Priority Data
Sep. 4, 2003 (CA) 2439667 A1

(51) Int. Cl. A61M 37/00; A61B 8/08

(52) U.S. Cl. CPC A61M 37/0092; A61B 8/483

(57) ABSTRACT

The present invention relates to an improved method for accelerating restoration of blood flow in treatment of an acutely thrombosed coronary artery by employing real time transthoracic 3D ultrasonic volume imaging at or near the base of the heart, and/or proximate the basal aspect of the associated left ventricular regional wall motion abnormality. Ultrasonic pulses provided by 3D imaging uniquely and necessarily deliver ultrasound to a broad target volume to stimulate the coronary arteries (which are difficult to image with ultrasound, and comprise tortious three dimensional structures), in view to providing an agitative and clot disruptive effect to a hidden, culprit, thrombosed, coronary vessel. In the preferred embodiment an intravenous microbubble solution is concurrently administered with 3D ultrasound which creates a dramatic synergy in disrupting the culprit thrombosis. Further incorporation of intravenously administered thrombolytics and co-use of transthoracic low frequency sonic vibration massage along with 3D ultrasonic imaging and microbubbles (including whereby thrombolytics are contained within microbubbles) to expedite initial reflow and facilitate microvascular flow (in avoidance of the no-reflow phenomenon following epicardial vessel recanalization) are also discussed.

### CLAIM OF PRIORITY

The present application is a continuation in part of co-pending U.S. patent application Ser. No 13/986,252 filed Apr. 17, 2013 which claims priority to U.S. patent application Ser. No. 12/798,437 (now U.S. Pat. No. 8,870,796), filed Apr. 5, 2010 with an issue date of Oct. 28, 2014, which claims priority to now abandoned U.S. patent application Ser. No. 12/291,128 filed Nov. 5, 2008 which claims priority to U.S. patent application Ser. No. 12/218054 (now U.S. Pat. No. 8,734,368) filed on Jul. 11, 2008 with an issue date of May 27, 2014 which claims priority to now abandoned U.S. patent application Ser. No. 11/036, 386 filed on Jan. 18, 2005 which claims priority to U.S. patent application Ser. No. 10/902,122 (now U.S. Pat. No. 7,517,328) filed Jul. 30, 2004 with an issue date of Apr. 14, 2009, which claims priority to Canadian Patent Application No. 2439667 A1 filed Sep. 4, 2003. The contents of these applications are incorporated herein by reference in their entirety.

### FIELD OF THE INVENTION

This invention relates to non-invasive mechanical stimulation systems to enhance coronary reflow in the emergency treatment of heart attack.

### BACKGROUND OF THE INVENTION

A quarter million people experience ST Elevation Myocardial Infarction (STEMI) heart attacks annually, and one of the most important factors for recovery is quick treatment to restore coronary blood flow to prevent muscle death

While Primary Percutaneous Coronary Intervention (PPCI) or “angioplasty” is the preferred treatment for STEMI, patient's are expected to receive intervention within 90 minutes of presentation to medical personnel, which even experienced centers struggle to accomplish. The search for non-invasive systems to accelerate early reperfusion in STEMI, preferably as an adjunct to intravenous thrombolytic drug delivery and/or intravenous administration of microbubbles (all of which can advantageously be delivered in ambulance while en-route to hospital or pre-cath), have therefore been sought after, but have been elusive.

Externally delivered ultrasound has been shown in-vitro and in animal models to disrupt thrombosis in view to clearing a thrombosed artery, however in these incidences the location of the clot was often known (with the ultrasound transducer placed directly over the clot), and/or the target artery was relatively superficial near the skin of the test subject. Reliable targeting of an acute coronary thrombosis (which is deep and a hidden target within the thoracic cavity) however is not presently possible with modern non-invasive ultrasonic imaging techniques.

Transcutaneous transthoracic (across the chest wall) ultrasound in combination with intravenously (IV) administered thrombolytics and/or microbubbles (e.g. microscopic lipid spheres)—whereby ultrasound enhances thrombolytic mixing and enzymatic action, while causing the microbubbles to resonate and cavitate making the bubbles very clot disruptive—has been more recently studied, but has struggled to demonstrate clinical promise in accelerating reperfusion in ST elevation myocardial infarction.

Part of the problem with ultrasound in coronary applications (particularly in humans, vs. animals such as small pigs—the bulk of which recent pre-clinical research is based on) is that the edges of the base of the human heart (wherein the coronary arteries arise and are substantially distributed) are interfaced and to a degree over ridden by lung which does not transmit ultrasound (unlike the pig—where the lung is posteriorly and inferiorly displaced away from the base of the heart). This coupled to a relatively thicker and more attenuating human chest wall (many STEMI patients are obese with poor acoustic windows) makes imaging and penetration of ultrasound towards an already elusive, hidden coronary vasculature, very unreliable. These obstacles were shown very apparently by the failure of the PLUS study (2003—results published in 2010), whereby nonspecifically directed ultrasound applied over the chest wall with low frequency non imaging ultrasound (27 kHz) in conjunction with thrombolytic drug therapy failed to enhance reperfusion in treatment of STEMI.

Therefore it has become apparent that transthoracic ultrasound to even potentially play a role in disrupting coronary thrombosis, requires a targeted imaging approach to ensure ultrasound is reaching key target areas of the heart which generally correlate to the anatomic position of the coronary vessels thereupon.

However it has been the Applicant's intuition that conventional use of two dimensional (2-D) imaging ultrasound, which comprises a singular scan plane of emitted ultrasonic beams (which the majority of STEMI trials following the PLUS study have and/or are presently utilizing), also, despite the advantage of targeting, is highly unlikely to provide an adequate stimulatory effect to a culprit coronary vasculature to assist in STEMI reperfusion, as 2-D ultrasound only enables small, minimized slices of ultrasonic dispersement which cannot reliably effect targeting along the length of a culprit, thrombosed coronary artery (which still remains a hidden target in ultrasonic imaging applications), and which is torturous and courses from the aortic root along the surface of the heart in a complex three dimensional (3-D) pattern. The uncertainty of ultrasound reaching the thrombosed coronary vessel is further complicated by the paucity of microbubbles and lytic agents which will actually flow into the thrombosed culprit vessel (because it is blocked with no flow).

To this end the present invention provides a long awaited solution in methodology to this long felt problem, in achieving optimized, accelerated emergency coronary thrombolysis and reflow by transthoracic ultrasound.

### SUMMARY OF THE INVENTION

The present invention relates to a new and improved method of transthoracically dispensing imaging ultrasound, preferably in the low Megahertz, >1 MHz frequency range (and at a high mechanical index, at least 1.2, preferably.>1.6), via real time 3-D imaging volume stimulation preferably targeting anatomic landmarks within the heart correlating to the proximal to mid aspects of the coronary vasculature to accelerate emergency reperfusion in STEMI.

The intuition of this invention rests on the realization that for non-invasive ultrasound to be effective as a clot disruptive modality to accelerate clearance of coronary thrombosis, it first must be targeted towards the heart by a skill based imaging technique (preferably proximate the base of the heart, and/or inclusive of the basal aspect of a left ventricular regional wall motion abnormality, wherein the coronaries and/or the likely site of culprit thrombosis likely arises) to establish an acoustic penetration window in avoidance of lung (which blocks ultrasound) , and in spite of an anticipated highly echo attenuating chest wall. Secondly, it must be realized that standard, single plane emitted 2-D imaging ultrasound (the still commonly studied approach in modern STEMI thrombolysis research) is not sufficient to provide mechanical stimulus to a culprit coronary vasculature, as 2-D ultrasonic acquisitions only enable a dispersal of ultrasound in thin slices which cannot stimulate to any meaningful degree the coronary arteries which remain as hidden targets, are hidden behind lung, and which elusively course from the aortic root with tortuosity in a complicated 3-D pattern.

Hence transthoracic ultrasound for disruption of coronary thrombosis should preferably be delivered by a skilled targeting technique via a cardiac 3D ultrasonic imaging transducer to image by real time volume (or at least real time multi-planar) acquisitions a basal to mid aspect of the heart including anatomic landmarks correlating to the locations of the coronary vessels; e.g. at the level of the aortic root—origin of the Left Main (LM) and Right Coronary Artery (RCA), left lateral aspect of mitral valve—mid Left Circumflex (LCx), rightward aspect tricuspid valve—mid RCA, leftward aspect of the pulmonary valve—distal LM, proximal LAD, proximal LCx), and including the intraventricular septum up to the papillary muscles (mid LAD and Posterior Descending Artery—PDA). This approach would best ensure an ultrasonic stimulus to include a substantial and meaningful portion of the proximal to mid coronary vasculature whereby the culprit hidden clot would most likely be situated. It should be emphasized that ultrasonic delivery to many of these target areas requires highly skilled imaging with a very experienced tech with a strong wrist as forceful angulation of the probe will be required, especially to view the; anatomic leftward aspect of the pulmonary valve (to capture a widow maker—distal LM, prox LAD) and the anatomic rightward aspect of the tricuspid valve (to capture the mid RCA), which are all challenging images (requiring a forceful steep angulation of the probe) to obtain in echocardiography!.

In the preferred embodiment, real time—continuously delivered 3-D volume ultrasonic acquisitions (towards or near the base to mid aspects of the heart, and at least including the aortic valve and/or aortic root—whereby the coronaries arise) should be administered along with IV thrombolytics (e.g. TPA, TNK) and an IV microbubble solution (such as Definity®, or Luminity®, EchoGen™ (Dodecafluoropentane emulsion, Albunex™, LEVOVIST™—Galactose-Palmitic Acid ultrasound contrast agent-, Air containing albumin microcapsules (Quantison™ and Myomap™), SonoVue™, Sulfurhexafluoride and Perfluorocarbon—containing microbubbles—Perfluorocarbon exposed sonicated dextrose albumin PESDA), which readily accumulates at the level of the aortic root and perfuses into the coronary vasculature. The ultrasound technician should be given an imaging protocol depending whether it is felt that the left vs. right coronary system contains the culprit thrombosis, which is possible to determine in view of the 12—lead ECG (e.g. inferior STEMI, likely RCA—Anterior STEMI, likely LAD, Lateral STEMI likely LCx or Distal RCA).

If for example the LAD is a presumed culprit the technician should establish an acoustic penetration window including the leftward aspect of the pulmonary valve, and then employ 3-D imaging, and then slowly pan back and forth from the pulmonary valve to the basal to mid septum (up to and just beyond the hinge'point from an observed Regional Wall Motion Abnormality (RWMA) which further maximizes the target volume to ensure coronary thrombotic stimulation. The volume directed ultrasonic waves will cause resonation and cavitation of the microbubbles making them extremely agitative and clot disruptive (and will also accelerate enzymatic action of the thrombolytic towards the thrombosis site), and these effects will importantly and necessarily occur along the length of the treated coronary artery, which courses away from the aortic root, in a complicated 3-D fashion.

It should be clear that by 3D “volume” imaging acquisition this term is in reference to where a preferably real time acquired ultrasonic imaging frame intentionally provides ultrasound wave dispersal in not just a plane via a single scan line (with a minimized slice thickness, i.e. with a large X and Y axis and a minimized Z axis, as in standard 2D echo), but in a multi planar and preferably “pyramidal” fashion, where the scan lines from the transducer are intentionally emitted at differing angles—such as to produce an ultrasonic dispersal and acquisition for each imaging frame covering an intentionally larger and substantially increased “Z” axis—and hence intentionally encompassing or interrogating a “3D” territory (real time 3D transducers have piezoelectric elements arranged in a grid fashion to enable “Z” axis emitted scan planes and control). Hence when the instant application describes a 3D imaging or volume acquisition in a frame of imaging, it should be understood that this may more broadly include the term “multi-planar” imaging or acquisition in a frame of imaging. It should also be understood that while preferably therapeutic imaging ultrasound would be emitted in an infinite number of planes such as to encompass a pure and complete target volume according to the invention, the invention should also cover variants whereby for example a multi-planar approach may include a minimum of at least 2 acquired planes per frame (such as for example produced at 90 degree angles to one another), which requires use of a 3D ultrasound transducer and also adds an intentionally broad “Z axis” to the target imaged area. It is also conceivable that a 3D ultrasound transducer could be adapted to scan an imaging plane in a first acquisition frame, and then automatically (without motion of the probe per say) scan a second imaging plane in a second frame, whereby to the human eye and to the point of therapy (in view of a very fast frame rate) would essentially display and interrogate a 3D volume but with a slight temporal distortion in the 3D image, and this variation should also be included in the present invention. Furthermore, the term “4D” ultrasound is commonly in reference to “3D” ultrasound acquisitions where the imaging is taken and displayed in real time, so it should be understood that when the term “real time” 3D ultrasound or ultrasonic acquisitions is used this could also be equivalently referred to as “4D” ultrasound according to the invention. In other words, the term 4D ultrasound in industry refers simply to a moving 3D ultrasonic picture.

The combination of 3-D ultrasound and IV microbubbles in establishing restoration of coronary blood flow in heart attack applications yields an important synergy in treatment. Real time emitted 3-D ultrasound without microbubbles will stimulate clearance of acute arterial thrombosis very modestly (to an unmeasurable amount), and microbubbles without 3-D ultrasound (for example if only real time 2-D imaging ultrasound is utilized), will be unlikely to receive ultrasonic stimulation near the thrombosis site, hence will work poorly - and only if one gets “lucky”.

The preferred embodiment also includes the co-joint IV administration of a thrombolytic agent, whereby real time delivered 3-D ultrasound will synergistically enhance the mixing of the lytic into the coronary circulation, and accelerate the enzymatic fibrinolytic action of the thrombolytic towards thrombolysis.

It is also foreseen that a thrombolytic agent may optionally be carried or encompassed within the microbubbles, which will cavitate and disrupt (or rupture) when exposed to ultrasound to thereby releasing their lytic contents locally, proximate the culprit coronary thrombotic blockage. This could lead to decreased lytic dosages, which would hopefully reduce the incidences of cerebral bleeds.

It should also be stressed that transthoracic 3-D ultrasound targeted to the heart, preferably applied to the basal origins of the coronaries and including proximate the ischemic, hypo-akinetic segment, could also—if applied with IV microbubbles, assist in slow or no-reflow after STEMI following emergency thrombolysis or PPCI. The local release of nitric oxide under influence of ultrasound, results in an increased capillary diameter and thus improvement of local epicardial and microvascular perfusion. Furthermore, the temperature rise and creation of micro-jets under influence of microbubble cavitation likely influences the pathogenetic mechanism of no-reflow in the microvasculature.

In a variation, transthoracic low frequency vibration massage at an impact frequency of between 1-1000 Hz (and more preferably in the 20-120 Hz—the resonance frequency of the epi-myocardium—and most preferably including 50 Hz range) and with a displacement amplitude of at least 1 mm (most preferably in this application applied to the upper back, but may also be applied upon the chest wall) may be provided along with 3-D ultrasonic imaging, and/or microbubbles and/or thrombolytics, whereby the applicant has shown by testing that low frequency vibration also provides clot disruptive effects (such as to provide early reflow) and is importantly transthoracically penetrative to the entire heart without the necessity of skill based targeting towards the coronary vasculature.

Low frequency vibration is also known to invoke endogenous liberation of Nitric Oxide, which is a potent vasodilator, and is predicted to relax coronary spasm which is often concomitant (up to 50% of the time) at the site of coronary thrombosis. This would assist initial reflow of the culprit thrombosed coronary artery and would importantly assist penetration of microbubbles and lytics into the thrombosed region. Moreover, low frequency vibration, particularly if applied substantially during the diastolic phase of the cardiac cycle (i.e. i.e. in avoidance of the isovolumetric contraction phase during the left ventricular force building period of systole), is known to enhance left ventricular diastolic relaxation which thereby improves cardiac output (by Starlings Law) and coronary flow. Vibration's relaxation of the otherwise stiff ischemic myocardium will assist ultrasound and microbubbles in the promotion of microvascular reflow in avoidance of no-reflow post reperfusion of the epicardial coronary artery.

Low frequency vibration may be applied to the upper back via a vibratory massager device which the STEMI patient may recline against during transthoracic ultrasonic therapy. This is preferred as it is easily achievable in ambulance or the emergency room, and would not obstruct medical personal in obtaining an IV, administrating microbubbles and/or a thrombolytic drug agent, and having a skilled ultrasonic imaging technician utilize a 3-D echo transducer to provide real time volume acquisitions at or near the base of the heart including the aortic valve and/or aortic root. Alternatively low frequency vibration could be applied directly to the chest wall of the patient—and this could be best accomplished by a novel device whereby a low frequency vibration actuator enables oscillation of the engagement face of the 3-D ultrasonic imaging transducer (thereby making the 3-D probe's engagement face a percussive contact node—enabling imaging and the transthoracic application of low frequency vibration).

It is therefore an object of the present invention to provide methods in use of cardiac 3D ultrasound for accelerating reperfusion in STEMI, and promoting reflow immediately following emergency interventions (whether by thrombolysis or by PPCI). The methods are below appended.

A method for accelerating reflow in a patient experiencing an acute coronary thrombotic obstruction (e.g. STEMI, or more broadly any acute coronary syndrome), resulting in an ST elevation (or Non-ST elevation) myocardial infarction, comprising the step of obtaining a transthoracic 3D ultrasonic acquisition (or an ultrasonic imaging acquisition enabling a targeted 3-D dispersal of ultrasound) of the heart of said patient, whereby 3D imaging of said heart optimizes targeting and dispersal of ultrasound within a target volume best encompassing the coronary vasculature of said patient, thereby promoting acceleration of clearance of said thrombotic obstruction. It is appreciated that a 3D ultrasonic volume acquisition (with an intentionally enlarged “z” axis) according to the invention may comprise or may be described (at least in part) as a multi-planar acquisition, or real time 3D acquisitions or 4D acquisitions (i.e. a motion picture of 3d ultrasonic imaging).

The method further comprising the step of intravenously administering a microbubble solution at any time prior to termination of the obtaining a transthoracic 3D volume acquisition, whereby 3D imaging of said heart optimizes agitation of said microbubbles within a target volume encompassing the coronary vasculature of said patient, thereby promoting acceleration of clearance said acute coronary thrombotic obstruction.

The method wherein said ultrasonic imaging comprises real time 3D, or 4D ultrasonic imaging.

The method wherein said 3D ultrasonic volume acquisition selectively targets at least one of the base of the heart, aortic root, aortic valve and a basal aspect of a left ventricular regional wall motion abnormality.

The method, further comprising the step of intravenously administering a thrombolytic in conjunction with said microbubble infusion.

The method further comprising the step of administering transthoracic vibration with a serial impact frequency in the range of 1-1000 Hz and a displacement amplitude of at least 1 mm in conjunction with said ultrasound and microbubbles.

The method wherein said vibration is applied to the upper back of said patient.

The method wherein said vibration is applied by oscillation of the engagement face of a 3d ultrasound transducer enabling said 3d volume acquisition.

A method for restoring coronary blood flow to a patient being treated following an acute coronary thrombotic obstruction comprising the steps of; intravenously dispensing a microbubble solution to a patient experiencing a heart attack, and obtaining a 3D ultrasonic image targeting the base of the heart of said patient at any time during the administration of a microbubble solution, whereby said 3D ultrasonic image provides a volume target best incorporating the proximal aspects of the coronary vasculature of said patient thereby enhancing ultrasonic action and clot disrupting effect of said microbubbles within said coronary vasculature.

The method wherein said 3D ultrasonic image is delivered in real time, and includes at least one of the aortic root and a left ventricular wall motion abnormality.

The method further comprising the step of administering a thrombolytic agent to said patient at any point prior to termination of the obtaining a 3D ultrasonic image.

A method for assisting reperfusion in a heart attack, comprising the steps of; a) intravenously dispensing a thrombolytic drug to said patient, and b) obtaining a 3D ultrasonic image of the heart of said patient at any time during step a, whereby said 3D ultrasonic image provides a dispensement of ultrasound to a volume target best incorporating a thrombosed culprit coronary vasculature of said patient thereby improving the fibrinolytic effectiveness of said thrombolytic drug upon said acute coronary thrombotic obstruction.

The method further comprising the step of intravenously administering a microbubble solution to said patient (said microbubbles possibly containing a thrombolytic drug agent) at any point prior to termination of step b.

It is understood, that a manufacturer, distributor or marketing agent of; a 3D ultrasonic imaging transducer, microbubbles and/or lytic agents may provide instructions in use of the combinations of the two or three (as defined above, with their substantial equivalents) to promote clinical adoption and sale of their product.

It is thereby another object of the present invention to provide a method for constructing a treatment system for assisting reperfusion in heart attack, comprising the steps of; a) providing a thrombolytic drug to a care provider wishing to treat said patient, and b) providing instructions to said care provider to obtain a 3D ultrasonic image of the heart of said patient at any time during intravenous administration of said thrombolytic drug, the ultrasonic beams of said 3D ultrasonic image deemed to interact with the location of a culprit thrombosed coronary artery responsible for said heart attack, whereby said 3D ultrasonic image provides a dispensement of ultrasound to a volume target deemed to incorporate said thrombosed coronary artery of said patient thereby improving the fibrinolytic effectiveness of said thrombolytic drug upon said thrombosed coronary artery. Similarly a further object of the present invention is to provide a method of constructing a treatment system to enhance reflow in a patient during or following an acute coronary thrombotic obstruction, comprising a) providing microbubbles, and b) providing instructions for administering imaging directed 3D ultrasound to the heart of said patient in conjunction with use of said microbubbles, said instructions available to an operator wishing to use said microbubbles to assist in reflow of said patient. The method wherein said instructions indicate microbubbles of said treatment system contain at least one thrombolytic drug agent.

A method of constructing a treatment system to enhance reperfusion in a patient during an ST elevation myocardial infarction, comprising the steps of a) providing at least one of a thrombolytic drug agent and (or) IV administrable microbubbles to a care provider wishing to treat the patient, and b) providing instructions for administering a 3D ultrasonic volume acquisition to the heart of said patient in conjunction with use of said at least one of a thrombolytic drug agent and (or) microbubbles, said instructions made available to an operator wishing to use said at least one of a thrombolytic drug agent and (or) microbubbles to enhance reperfusion in said patient. The method further defined whereby a thrombolytic drug agent is contained within the microbubbles, whereby the microbubbles upon stimulation by ultrasound would rupture locally liberating the contents of the thrombolytic agent.

A method of constructing a treatment system to enhance myocardial perfusion in a patient experiencing at least one of ST elevation myocardial infarction (or more broadly and acute coronary syndrome) and slow to no reflow following emergency reperfusion treatment, comprising the steps of a) providing a ultrasonic imaging transducer capable of 3D imaging, and b) providing instructions for administering preferably real time 3D ultrasound to the heart of said patient via said transducer, said instructions made available to an operator wishing to use said imaging transducer to enhance myocardial perfusion in said patient. Or comparably a method of constructing a treatment system to enhance reperfusion in a patient during an ST elevation myocardial infarction, comprising the steps of a) providing a cardiac 3-d ultrasonic imaging transducer, and b) providing instructions for administering a 3D ultrasonic volume acquisition (or real time 3D volume acquisitions, or equivalently a 4D volume acquisition) to the heart of said patient in conjunction with use of intravenously administered microbubbles, said instructions available to an operator wishing to use said imaging transducer to enhance reperfusion in said patient.

### BRIEF DESCRIPTION OF THE DRAWINGS

The apparatus and method of the present invention will now be described with reference to the accompanying drawing figures, in which:

FIG. 1 shows a diagrammatic illustration of a 3-D transthoracic echo transducer imaging and treating the basal to mid regions of a heart (including coronary thrombosis marked by “X”) by emission of a pyramidal 3-D ultrasonic imaging volume according to the preferred embodiment of the invention.

FIG. 2 shows a diagrammatic illustration of a conventional 2-D slice ultrasonic image with ultrasonic stimulated microbubbles at the level of the aortic valve according to the common wisdom in the art. Only the origin of the left main and right coronary artery are barely shown and hence receiving treatment.

FIG. 3 in contrast shows a diagrammatic illustration of a 3-D ultrasonic volume acquisition with ultrasonic stimulated microbubbles at the level of the aortic root to base to mid aspect of the heart according to the invention. Here a substantial portion of the proximal to mid aspects of the coronary vasculature including the site of thrombus (which remains a hidden target in clinical imaging) receives treatment.

FIG. 4 is a perspective view of a STEMI patient receiving real time 3-D volume ultrasonic imaging acquisitions while receiving IV infusions of microbubbles and thrombolytic therapy to enhance coronary epicardial recanalization and reflow according to the preferred embodiment of the invention.

### DETAILED DESCRIPTION

Coronary thrombosis on a ruptured coronary plaque is the main pathophysiologic event that leads to acute coronary syndromes. Current recanalization therapies in these disease states include pharmacological thrombolysis and PPCI, both of which have improved the prognosis of patients with STEMI. Each of these therapeutic interventions, however, has significant limitations. The time required to open a coronary vessel successfully with PPCI, even at the most experienced centers, is often greater than 90 minutes after presentation to the Emergency Department, during which extensive myocardial necrosis may have already occurred. Reperfusion using IV thrombolytics (which can be administered quickly, pre-hospital) is most effective if given within the first hour after the onset of symptoms in STEMI, but effective epicardial recanalization is achieved in less than 60% of patients. Furthermore, the doses of thrombolytics utilized in clinical trials have increased the risk for intracerebral hemorrhage, even if patients with previous stroke are excluded. Finally, neither PPCI or thrombolytic agents have reduced the risk for microvascular no reflow, a phenomenon in which there is a persistent perfusion abnormality within the risk area even after epicardial recanalization. This phenomenon correlates with lack of ST segment resolution on the 12 lead EKG, and is associated with post-infarction complications.

The use of transthoracic ultrasound, particularly in conjunction with IV thrombolytic drug agents, and IV microbubbles (which in tandem work co-operatively to agitate and disrupt coronary thrombosis, to assist in epicardial recanalization and reflow) have thereby emerged in the field of emergency STEMI treatment.

The history of transthoracic ultrasound to accelerate reperfusion in STEMI has evolved over the years with steps of intuition gleaned following missteps and multiple failures. The first clinical trial (The PLUS study, TIMI3 systems Inc.—2003—results published in 2010), utilized a low frequency ultrasound transducer (27 khz, non-imaging) placed over the chest wall of a STEM! patient (without specific targeting of the ultrasound beams, to ensure hitting key regions of the heart!) along with IV administration of IV thrombolytics. As expected in light of the Applicant's disclosure, this trial failed to enhance epicardial coronary recanalization.

The researchers of the PLUS study failed to recognize that for transthoracic ultrasound to reliably reach the heart (and let alone a hidden culprit coronary vasculature), a targeted imaging approach (with a small foot print imaging probe enabling skilled inter-ribspace force and angulation) would as a minimum be required to navigate the ultrasound away from lung (which does not transmit ultrasound) and across an identified acoustic penetration window (in view of a highly attenuating human chest wall—particularly for STEMI applications where a significant portion of patient's are obese).

Transthoracic imaging and treatment ultrasonic systems were reported by the Applicant (see parent application, US 20060025683, filed January 2005, incorporated herein in entirety by reference), partly in response to the PLUS study failure, presenting numerous imaging directed solutions particularly useful and necessary to accelerate reperfusion in cardiac applications. But what is even more interesting, it will be shown in light of the Applicant's disclosure that even standard 2-D echocardiograhic imaging via a skilled approach (to ensure ultrasonic targeting of the heart, and even a hypokinetic region) will not be enough—a further inventive step (i.e. use of “3-D” ultrasound) must be realized.

The invention will now be described in view of the appended figures.

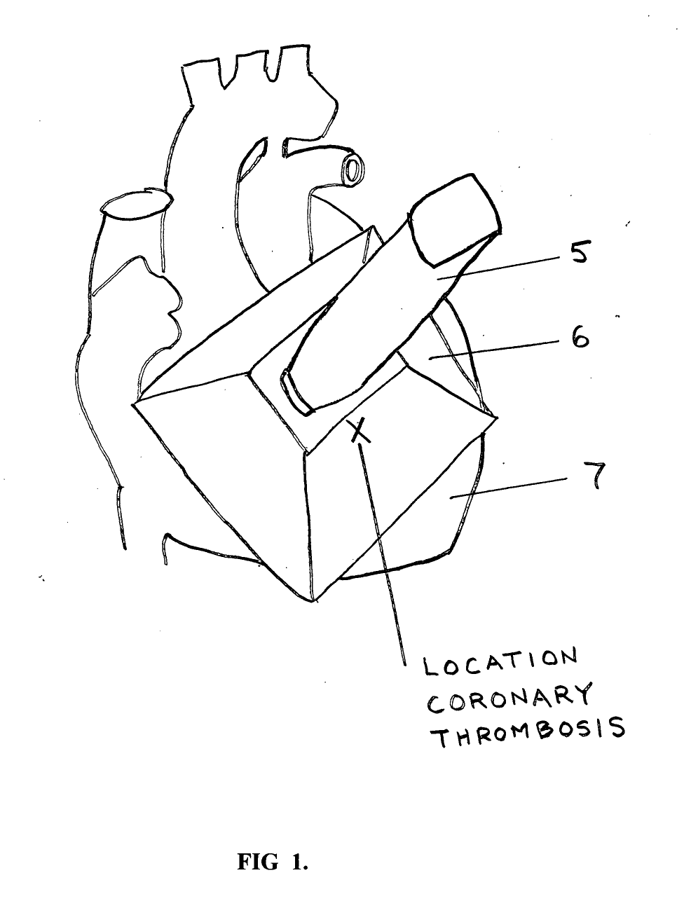

FIG. 1 diagrammatically depicts a 3-D ultrasonic transducer 5 emitting a pyramidal 3-D volume ultrasonic imaging dispensement 6 directed over a heart 7 with acute coronary thrombosis—not shown (the location of which marked by “X”)—, according to the preferred imaging and treatment method of the invention. Co-administration of microbubbles and thrombolytic drug therapy is not shown. It is understood that transducer 5 would be positioned and angled upon a STEMI patient's chest wall to achieve an acoustic penetration window which enables penetration of 3-D ultrasonic dispensement 6 to include at least the basal, and preferably basal to mid regions of key regions of heart 7, to capture the anatomic location of the proximal to mid coronary vasculature which overlies this portion of the heart. It should be appreciated that 3-D volume ultrasound imaging dispensement 6 is preferably delivered continuously in real time (hence providing agitation of microbubbles over a period of time and enabling viewing of a 3D ultrasonic image as a motion picture (or “4D” scanning)—which cannot be appreciated on the still image of FIG. 1.

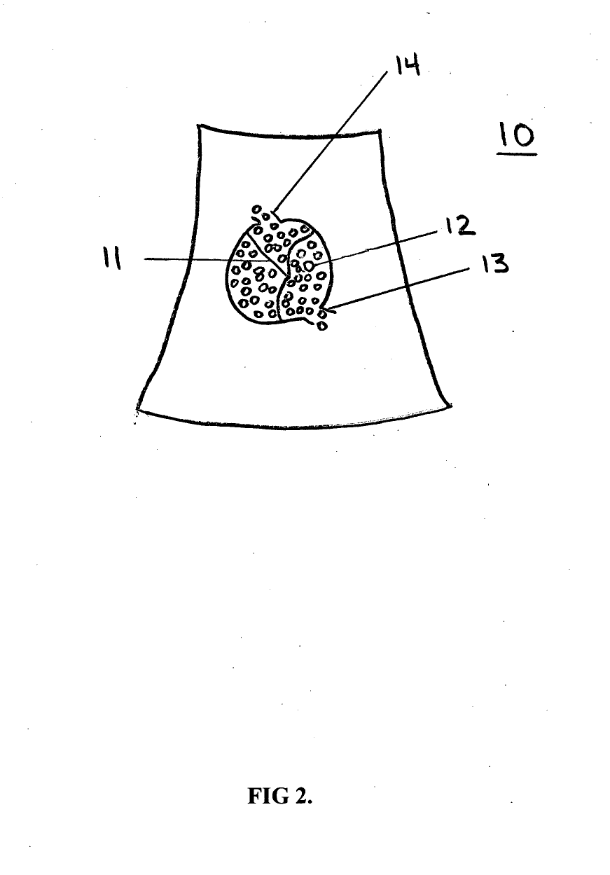

FIG. 2 diagrammatically depicts a transthoracic ultrasound 2-D slice acquisition 10 showing the aortic valve 11, with ultrasonically stimulated microbubbles 12. Stimulated microbubbles 12 are seen to enter the origin of the left main 13 and right coronary 14. Note how the coronary vasculature territory included and stimulated by therapeutic imaging ultrasound (and resonating microbubbles) is bare minimal, missing the entire coronary tree and culprit thrombus (not shown) which resides beyond the vessels origins.

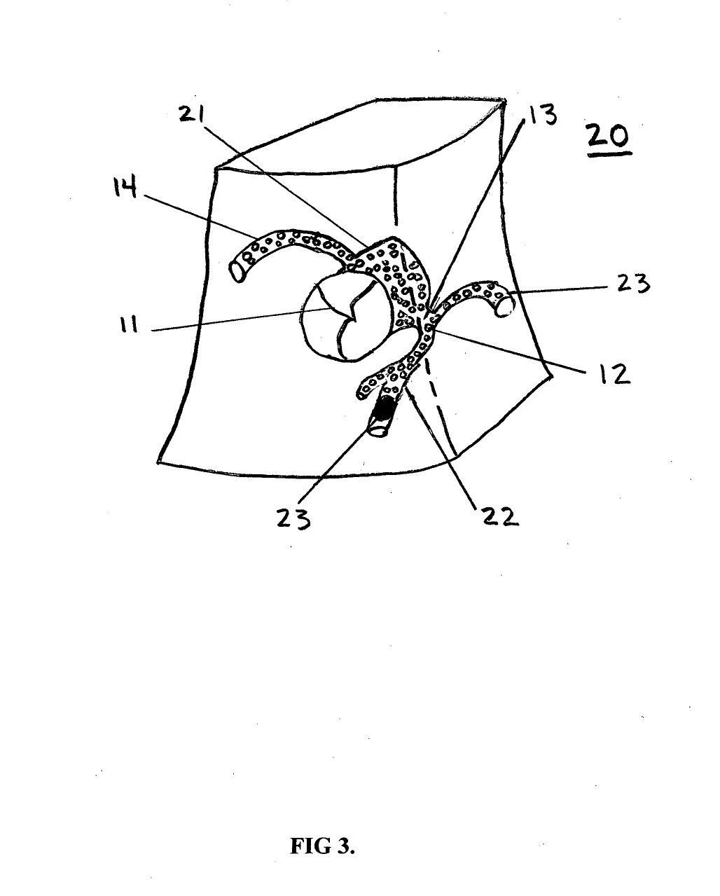

FIG. 3 in contrast diagrammatically depicts a transthoracic ultrasound pyramidal 3-D volume acquisition 20, showing the aortic valve 11, aortic root 21, entire left main 13, proximal to mid left anterior descending artery (LAD) 22, left circumflex 23, and proximal to mid aspect of the right coronary artery 14; with ultrasonically stimulated microbubbles 12. Note how the coronary vasculature territory included and stimulated by therapeutic imaging ultrasound (and resonating microbubbles) is now much more substantial, including capture of the proximal to mid coronary vasculature, and including agitation of microbubbles at the site of coronary thrombosis 23, shown in the proximal segment of the LAD. Note, while thrombus 23 is shown in this image, this is merely shown diagrammatically for educational purposes (i.e. to show that a volume directed ultrasonic imaging beam would be likely to pass through a location of a culprit coronary thrombus). As stated earlier, the small coronary lumen containing thrombosis cannot be imaged by echocardiography (hence remains a hidden target) and this is true regardless of use of 2-D or 3-D echo. It should be appreciated again that a series of 3-D volume acquisitions 20 are preferably delivered continuously in real time (hence providing agitation of microbubbles over a period of time and enabling viewing of a 3D ultrasonic image as a motion picture (or “4D” scanning)—which cannot be appreciated on the still diagrammatic image of FIG. 3.

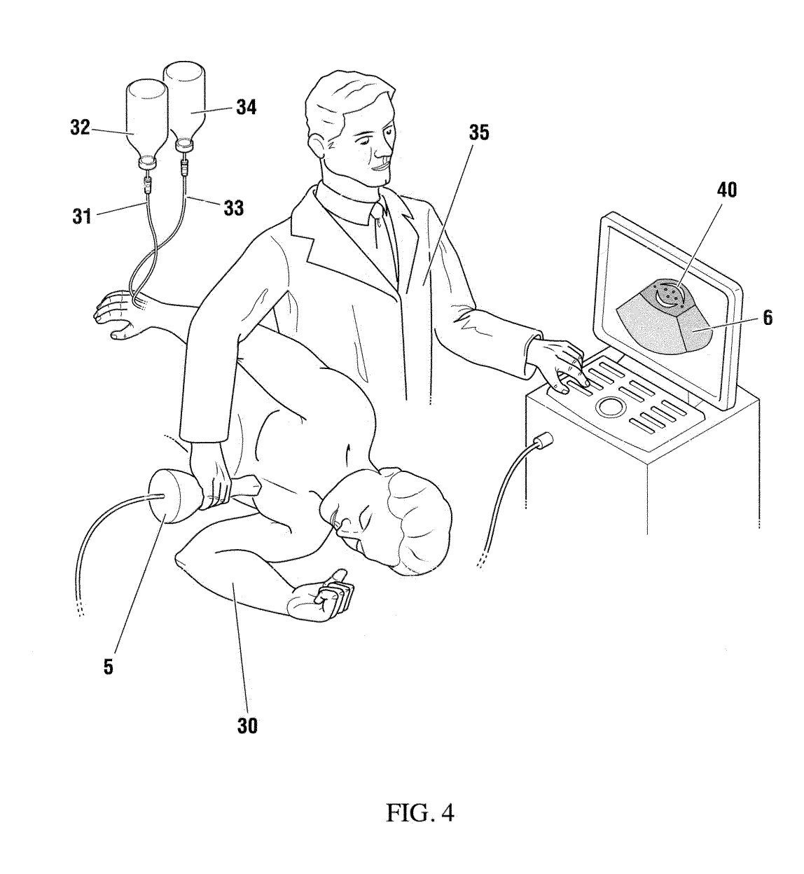

FIG. 4 shows a perspective view of the preferred embodiment according to the invention. STEMI patient 30 is shown with an IV line 31 enabling administration of a microbubble solution 32. A second IV line 33 concurrently enables delivery of a thrombolytic agent 34. Echo operator 35 by use of 3D ultrasonic imaging transducer 5 is shown locating a real time 3-D image 40 (note presence of microbubbles shown as fine dots in the aortic root), with leading edge at the level of the aortic root (and thereby including a 3-D volume ultrasonic imaging dispensement 6 encompassing a broad aspect the aortic root to the base to mid epicardial regions of the heart to maximize inclusion of the proximal to mid coronary vasculature.

In a preferred variation a STEMI patient may also recline against a low frequency vibration mat (not shown) preferably operable to emit percussion to the upper back of a STEMI patient with an impact frequency in the 20-120 Hz range (i.e. the resonance frequency of the epi-myocardium of the heart) via a vibration actuator embedded within the mat. Vibrations are preferably emitted during the diastole of the patient (in avoidance of the isovolumetric contraction period), which has been shown to enhance diastolic relaxation and coronary flow, to help stimulate initial epicardial arterial recanalization (by agitating thrombosis, improving mixing of systemically delivered medicants into the blocked otherwise zero flow culprit circulation, and relieving coronary spasm often present at the thrombosis site) and prevent or treat no-reflow (primarily by enhanced relaxation to the ischemic myocardium and stimulated vasodilatory nitric oxide release within the micro coronary vasculature).

In a typical situation a confirmed STEMI patient, on arrival at the hospital or during initial ambulance transport receives ultrasound contrast agent Luminity® during a simultaneously pulsatile 3D ultrasound application using a diagnostic ultrasound machine (e.g. iE33—Philips, Best, the Netherlands) with a frequency of 1.6 MHz and a mechanical index of 1.6. In this case a proximal coronary occlusion was predicted by initial ECG wherein the ST elevation was greater than 6 mm by sum of leads. The 3D probe is used to obtain 3D full volume images of the aortic root in the parasternal short-axis view to ascertain that at least the proximal parts of the epicardial coronary artery system are encompassed within the target volume treatment/imaging zone. In order to ensure that the microbubbles replenish around the occlusion of the infarct-related artery, ultrasound is applied intermittently (5 s on, 5 s off). The microbubbles are infused intravenously for 15 min using a continuous infusion pump at a rate of 200 ml/h. The patient is then sent to cath whereby epicardial recanalization of the culprit artery has already occurred.

In another example, a patient with crushing chest pain calls 911 whereby an ambulance is dispatched and 12-lead ECG shows marked ST elevation in the anterior leads (hence predicting a clot in the left coronary system—likely the proximal or mid LAD). Quick echocardiographic inspection shows an obvious Regional Wall Motion Abnormality (RWMA) involving the anterior septum which extends to the apex. The hinge point to this RWMA is identified to involve the proximal portion of the septum, hence 3-D ultrasonic imaging is initiated centering at the hinge point along with low dose thrombolytic and microbubble administration. The echo technician slowly angles the probe slightly distal and proximal from the hinge point and up to the leftward aspect of the pulmonary valve and including the aortic root to further broaden the target 3 D volume along the anatomic territory likely to underlie the entire length of the LAD. Restoration of ST segment and improvement of wall motion abnormality occurs—promptly, and the patient is referred for cardiac catheterization within 24 hrs of hospital admission.

In this case real time 3-D volume acquisitions were preferably utilized for imaging and treatment, but as an alternative imaging and targeting could also have been accomplished by limited 3-D, or multiplane or biplane imaging (i.e. showing long and short axis of the intraventricular septum), which would also improve the likelihood of targeting a culprit thrombosis site (vs. mere 2-D slice imaging) along the LAD.

In another example a patient presents with acute inferior wall STEMI with ST elevation in lead V 4 R (indicating right ventricular infarction, likely from a thrombotic occlusion involving the proximal Right Coronary Artery (RCA) thereby also blocking flow to the RV marginal branch. In this case the echo technician will emphasize parasternal window 3D imaging (preferably short axis) to include as much as possible of the aortic root—to visualize the proximal RCA, and then switching to 3-D mode to slowly pan from the aortic root to the anterior and rightward aspect of the tricuspid valve which follows the course of the proximal to mid RCA. IV microbubbles and low dose thrombolytic therapy is initiated concurrent with 3-D ultrasonic administration to hasten reperfusion.

In another example, a STEMI patient has received an emergency coronary stent to the proximal LAD, but there is only TIMI 1 to 0 reflow (i.e. slow to no—reflow). Despite intracoronary administration of various drugs, TIMI flow remains poor, and ST elevation remains present. The patient is taken off the cathlab table to the holding area whereby 3D ultrasonic imaging centered at the regional wall motion abnormality associated with the infarct commences along with intravenous administration of microbubble solution. Diastolic low frequency vibration is also applied to the patient's upper back. ST segment resolution is shortly realized thereafter.

The applicant explixitly published the method of utilizing “3-D” ultrasonic imaging targeted towards a basal aspect of the heart to encompass the coronary vasculature in a treatment system to accelerate reperfusion in STEMI in US patent application #20060025683 filed Jan. 18, 2005 (see pg. 5, par. 0040, right column 19 lines down in view of pg. 4, par. 0042, line 7, in view of pg. 2 par. 0018) which is a parent family member to the present instant continuation in part application. That the application could be co-administered with intravenously administered microbubbles and/or a thrombolytic agent was also described in this parent filing (see pg. 3 par. 20 and pg. 4, par 0036, left column, 10 lines down—respectively). That low frequency vibration could be transthoracically administered as adjunct to the above is also clearly depicted as a central them to this and subsequent grand-parent filings. Hence the inventive date according to publication relating to this continuation in part filing should coincide with at least the inventive date of this parent filing.

The evolution of thinking in approach to using ultrasound to disrupt thrombosis, and eventually leading into pre-clinical and clinical STEMI trials, underscores the non-obvious nature of the Applicant's invention in use of 3-D ultrasonic imaging.

Below are a list of studies related to use of ultrasound to disrupt thrombosis published before and slightly after the Applicant's invention.

Tachibana (1995): in-vitro—170 kHz (low frequency non imaging ultrasound) targeted directly over clot, improved thrombolysis with lytics and microbubbles. Note, low frequency ultrasonic “kHz” ultrasound (less than about 1 MHz) cannot be used to effect diagnostic imaging, but has been shown in-vitro to have a stronger penetration and clot disruptive effect than MHz (imaging) ultrasound. That is why non-imaging kHz ultrasound thrombolysis studies were of particular interest during this period.

Porter (1996): in-vitro—20 kHz (low frequency non imaging ultrasound), targeted directly over clot, increased thrombolysis with microbubbles, optimized with thrombolytics and microbubbles.

Nishioka (1997): canine illio-femoral artery 20 kHz (low frequency non imaging ultrasound) targeted directly over a superficially located clot, increased clot disruption with microbubbles.

Kondo (1999): in-vitro—10 MHz, imaging frequency ultrasound directed directly over clot, increased clot disruption with microbubbles.

Birnbaum (1998): rabbit iliofemoral artey—37 kHz (non-imaging ultrasound without lytics), targeted directly over a superficially located clot—according to the author “The ultrasound transducer was applied transcutaneously over the arterial occlusion site, which was marked on the skin with a metallic marker that was positioned at the time of angiography”.—increased recanalization rate with mirobubbles.

Siegel (2001): small canine coronary artery (mid left anterior descending), 20 kHz (non imaging ultrasound), targeted directly over known location of clot (canine, thin chest wall—non representative to humans and medial location of clot, away from lung), enhanced reflow.

Cohen and Siegel (2003): first phase 1-2 human STEMI trial with transcutaneous ultrasound! 27 kHz (non-imaging ultrasound), shows safety and feasibility of the technique in 25 patients. The Applicant (a cardiac echo tech and cathlab tech) realized well before this study that non directed ultrasound will not reliably reach the human heart, and particularly key areas of the heart, as the lung covers a substantial amount of the coronary circulation (air does not transmit ultrasound!), and the human chest wall is denser and more highly attenuating to ultrasound than in small pig or canine model.

Applicant files (Sep. 2, 2003) his first vibration and ultrasonic thrombolysis patent application, CA 2439667 A1, (priority grandparent document to this present filing), which emphasized use of low frequency vibration in the 1-1000 Hz range (which penetrates to all aspects of the heart without need for targeting) along with a therapeutic transthoracic ultrasound delivery, including volumetric dispersals of theraepeutic ultrasound (targeted by imaging ultrasound). Use of microbubbles and thrombolytic therapy as an adjunct to vibration and ultrasound was further listed.

Hudson and Sigel—Plus study (2003—results published in 2010): first phase 3 human STEMI trial with transcutaneous ultrasound! 27 kHz (non imaging ultrasound, applied over the chest wall. As predicted in light of the Applicant's dislcosure—no enhancement of reperfusion with IV thrombolytics!!.

Applicant files (January 2005) second patent application (US20060025683) reinforcing teachings that for cardiac applications transthoracic imaging is required to target ultrasound to the heart for STEMI applications. Multiple solutions are provided, explicit use of “3D imaging” ultrasound for STEMI published, preferably with thrombolytic and microbubbles.

Slikkerveer (2008, results published 2011): human STEMI trial, 1.6 MHz 3D ultrasound (the Applicant's invention is tested!), 3X greater reperfusion rate 3D ultrasound with microbubbles and low dose thrombolytic! Larger trials being worked on for FDA approval.

Xie (2009): pig coronary, LAD occlusion, 1.5 MHz 2-D ultrasonic imaging, known location of clot, recanalization rates of 53 to 60% within 90 minutes of administration of therapy (which disappointedly was not better than the clinical benchmark of IV thrombolytic therapy without ultrasound for STEMI in humans, which is 50-60%). Note in this study the location of the clot was again known (so the target area—the intraventricular septum—was advantageously focused upon), and again the clot was medially placed and away from lung (in the pig the lungs are inferiorly and posteriorly displaced away from the base of the heart), and in a fairly small animal—average weight only 36 kg with a noon representatively thin chest wall). Of interest Xie and his associates chose to study “2-D” single plane slice ultrasound, which suggests that use of 3D ultrasound remained at least at this time non-obvious to those skilled in the art during conception of the study. Researcher's in this trial admit in hindsight (perhaps in light of the Applicant's invention) that “A three-dimensional application of high MI impulses may improve the likelihood that the entire volume of the RA [risk area] and upstream coronary artery are being insonified.”

Mathias (2015): human phase 1-2 STEMI trial underway, however with again 1.7 MHz “2-D” slice ultrasonic imaging!—likely following the pre-clinical testing of Xie (above). Mathias and his co-workers are trusting results in small pigs, with thin chest walls and where again the location of clot was known, and the lungs (unlike in humans) do not block the coronary vasculature. This further underscores the non-obvious nature of the Applicant's invention which emphasizes that 3-D ultrasound is required for humans, where the exact clot location will be unknown, there is a thicker more attenuating chest wall, and where the lungs overly key areas of the base of the heart making formidable, attenuating obstacles.

As can be seen above, transthoracic ultrasonic therapy as means for enhancing reperfusion in STEMI (with or without microbubbles and thrombolytic drug therapy) has up until and even following the Applicant's invention struggled (and will apparently continue to struggle) to gain medical acceptance, either because of a lack of imaging directed ultrasonic targeting, misleading pre-clinical animal trials, and importantly the lack of use of 3-D imaging volume acquisitions with appropriate echocardiographic imaging protocols. Tests are just now confirming that transthoracic 3-D ultrasound of the basal regions of the heart, preferably with co-use of IV microbubbles and thrombolytic drugs (i.e. Sonolysis Trial, Netherlands—where three times the number of STEMI patients reperfused when given 3D cardiac ultrasound), are proving effective to enhance coronary reflow and patient's clinical outcomes in emergency STEMI treatment. The Applicant's invention represents therefore what has been a non-obvious long sought after solution to a long felt need.

As will be immediately apparent to those skilled in the art in light of the foregoing disclosure, many alterations and modifications are possible in the practice of this invention without departing from the spirit or scope thereof. Accordingly, the scope of the invention is to be construed in accordance with the substance defined, and as described, by the following claims.

1. A method for promoting reflow in a patient experiencing an acute coronary thrombotic obstruction, comprising the step of;
a) obtaining a 3D ultrasonic imaging acquisition of the heart of said patient,
whereby 3D ultrasonic imaging of said heart enables targeting of a 3D dispersal of ultrasound encompassing a culprit coronary vasculature of said patient, thereby promoting clearance of said thrombotic obstruction.

2. The method of claim 1, further comprising the step of intravenously administering a microbubble solution at any time prior to termination of step a,
whereby said 3D dispersal of ultrasound promotes agitation of said microbubbles within said culprit coronary vasculature of said patient.

3. The method of claim 2, wherein said ultrasonic imaging comprises real time 3D, or 4D ultrasonic imaging.

4. The method of claim 2, wherein said 3D ultrasonic imaging acquisition includes an image of at least one of the base of the heart, aortic root, aortic valve and a left ventricular regional wall motion abnormality.

5. The method of claim 2, further comprising the step of intravenously administering a thrombolytic in conjunction with said microbubble infusion.

6. The method of claim 1, further comprising the step of administering localized, transthoracic vibration with a serial impact frequency in the range of 1-1000 Hz and a displacement amplitude of at least 1 mm in conjunction with said 3D dispersal of ultrasound.

7. The method of claim 2, wherein said 3D ultrasonic imaging acquisition comprises at least one of a volume acquisition and a multi-planar acquisition.

8. The method of claim 6, wherein said vibration is applied by oscillation of the engagement face of a 3d ultrasound transducer enabling said 3D ultrasonic imaging acquisition.

9. A method for restoring blood flow within a thrombosed coronary vasculature of a patient comprising the steps of;
a) intravenously dispensing a microbubble solution to said patient, and
b) obtaining an ultrasonic imaging acquisition enabling a 3-D dispersal of ultrasound targeted towards the heart of said patient at any time during step a,
whereby said 3-D dispersal of ultrasound provides a volume target deemed to incorporate said thrombosed coronary vasculature of said patient thereby promoting the clot disrupting effect of said microbubbles within said thrombosed coronary vasculature.

10. The method of claim 9, wherein said 3-D dispersal of ultrasound enables acquisition of a real time 3D ultrasonic image including at least one of the aortic root, a left ventricular wall motion abnormality, and an anatomic landmark deemed proximate said thrombosed coronary vasculature.

11. The method of claim 9, further comprising the step of administering a thrombolytic agent to said patient at any point prior to termination of step b.

12. A method for constructing a treatment system for assisting reperfusion in a heart attack, comprising the steps of;
a) providing at least one of a thrombolytic drug and intravenously administrable microbubbles to a care provider wishing to treat a patient experiencing said heart attack, and
b) providing instructions to said care provider to utilize a 3D ultrasonic imaging of the heart of said patient at any time during intravenous administration of said at least one of said thrombolytic drug and said microbubbles, wherein the ultrasonic beams enabling said 3D ultrasonic image are deemed to interact with the location of a thrombosed coronary vasculature responsible for said heart attack, and
whereby said 3D ultrasonic imagine enables a 3D dispensement of ultrasound to promote the effectiveness of said at least one of a thrombolytic drug and microbubbles in restoration of blood flow within said thrombosed coronary vasculature.

13. The method of claim 12, wherein said microbubbles contain said thrombolytic drug agent.

14. A method of constructing a treatment system to enhance reflow in a patient following diagnosis of an ST elevation myocardial infarction comprising the steps of;
a) providing microbubbles, and
b) providing instructions for administering 3D ultrasonic imaging of the heart of said patient in conjunction with use of said microbubbles, said instructions made available to an operator wishing to use said microbubbles to assist in reflow of said patient.

15. The method according to claim 14, wherein said microbubbles contain a thrombolytic drug agent.

16. A method of constructing a treatment system to enhance reperfusion in a patient during an acute coronary syndrome, comprising the steps of
a) providing a cardiac 3-d ultrasonic imaging transducer, and
b) providing instructions for acquiring a 3-d ultrasonic image of the heart of said patient by use of said 3-d ultrasonic imaging transducer in conjunction with an intravenous administration of at least one of microbubbles and a thrombolytic agent, said 3-d ultrasonic imaging transducer enabling targeting of a 3-d dispensement of ultrasound towards a culprit coronary vasculature of said patient thereby promoting the effectiveness of said at least one of microbubbles and a thrombolytic drug agent towards restoration of blood flow within said culprit coronary vasculature, and,
c) making said instructions available to an operator wishing to use said imaging transducer to enhance reperfusion in said patient.
